(19) United States
(12) Patent Application Publication
(10) Pub. No.: US 2016/0113574 A1
(43) Pub. Date: Apr. 28, 2016

(54) ELECTRONIC DEVICE FOR CALCULATING PHYSICAL ENERGY CONSUMPTION

(71) Applicant: Ossong International Co., Ltd., Hsinchu City (TW)

(72) Inventors: Chang-Yo HSUAN, Hsinchu City (TW); Hsin-Kuo HO, Hsinchu City (TW)

(21) Appl. No.: 14/522,305

(22) Filed: Oct. 23, 2014

(51) Int. Cl. A61B 5/00; A61B 5/11

(52) U.S. Cl. CPC A61B 5/4866; A61B 5/1118; A61B 5/7278; A61B 5/7475; A61B 5/742

(57) ABSTRACT

Provided discloses an electronic device for calculating physical energy consumption including a gravity sensing module, a storage module, a processing module, and a touch control display module. The gravity sensing module detects at least one movement of the electronic device for calculating physical energy consumption to generate at least one movement signal. The storage module stores a plural physical training mode and a plural predetermined exercise value. The processing module connects to the gravity sensing module and the storage module, the processing module receives at least one movement signal and generates at least one active value depending on the movement signal, and subtracts at least one active value from the predetermined exercise value corresponding to one of the physical training modes to generate at least one exercising consumption signal. The touch control display module connects to the processing module, and receives and displays at least one exercising consumption signal.

### BACKGROUND OF THE INVENTION

1. Field of the Invention

The present invention relates to an electronic device, in particular with respect to an electronic device for calculating physical energy consumption.

2. Description of the Related Art

Owing to the people live a fast-paced life and have tremendous work stress, many chronic diseases are easy to be formed and the weight problem is increased gradually. Consequently, maintaining regular and continuous exercise has caused a lot of attentions to the public and has been regarded as the principle of keeping physical and mental health, and as to female, that reaches to purpose of weight reduction.

In order to maintain in slim shape, female not only keeps doing exercise, but also calculating the related statistic of diet and exercise, such as calorie, records of cardiopulmonary function, provided energy of food and so on to attain to the best body shaping effect.

However, calculations of daily exercise amount as well as the provided energy of daily meals are of a mass of statistic data and algorithm thereof is very complicated. In addition, even the statistic data may be executed by computer or smart phone, the user still has to categorize or aggregate the calculated numeric values by operating complicated software program so that the exercising consumption or calorie for a day may therefore be obtained.

As a result, inventor of the present invention ponders and then designs an electronic device for calculating physical energy consumption which aims to improve the defects of the current technique so as to increase the industrial practicability.

### SUMMARY OF THE INVENTION

In view of the aforementioned problems of the prior art, purpose of the present invention is to provide an electronic device for calculating physical energy consumption to overcome the existing shortcomings.

According to the purpose of the present, it may provide an electronic device for calculating physical energy consumption comprising a gravity sensing module, a storage module, a processing module and a touch control display module. The gravity sensing module may detect at least one movement of the electronic device for calculating physical energy consumption to generate at least one movement signal. The storage module may store a plurality of physical training modes and a plurality of predetermined exercise values. The processing module may connect to the gravity sensing module and the storage module, the processing module may receive the at least one movement signal and generates at least one active value depending on the movement signal, and may subtract the at least one active value from the predetermined exercise value corresponding to one of the physical training modes to generate at least one exercising consumption signal. And the touch control display module may connect to the processing module, and may receive and displaying at least one exercising consumption signal.

Wherein, the touch control display module may detect at least one touch to generate at least one touch signal, and the processing module may receive the at least one touch signal to accordingly conduct switching in the plurality of physical training modes.

Wherein, the electronic device for calculating physical energy consumption may further preferably comprise a power module connecting to the gravity sensing module, the storage module, the processing module and the touch control display module; the power module providing necessary power to the gravity sensing module, the storage module, the processing module and the touch control display module.

Wherein, the electronic device for calculating physical energy consumption may further preferably comprise a capacity module connecting to the power module, the capacity module generating electrical energy according to the movement of the electronic device, and the capacity module outputting the electrical energy to the power module.

Wherein, the processing module may generate a two dimensional barcode pattern according to the exercising consumption signal, and the two dimensional barcode pattern may be displayed by the touch control module for image capturing by an external electronic device, and the exercising consumption signal may be stored in the external electronic device.

Wherein, the electronic device for calculating physical energy consumption may be detachably connected to an external stationary device.

Wherein, the electronic device for calculating physical energy consumption may further preferably comprise a wireless transmission module connecting to the processing module, the wireless transmission module receiving the exercising consumption signal and transmitting the exercising consumption signal to an external electronic device.

Wherein, the exercising consumption signal may be letter, diagram or combination thereof.

Wherein, the storage module may further store a plurality of user modes providing setting for a user, and the processing module may determine the predetermined exercise value corresponding to each of the physical training modes according to the plurality of user modes.

Wherein, when the touch control display module detects at least one touch and generates at least one touch signal, the processing module may receive the at least one touch signal to accordingly conduct switching in the plurality of user modes.

In conclusion, the electronic device for calculating physical energy consumption according to the present invention may have one or more advantages as follows:

(1) The electronic device for calculating physical energy consumption may detect the movement generated by the electronic device for calculating physical energy consumption which wears by the user through the gravity sensing module to calculate the exercising consumption amount so as to provide the user for the convenience of acquiring the self-exercise consumption amount and resolve the problems concerning the user has to calculate the self-exercise consumption amount in the prior art.

(2) By the capacity module generating electronic energy, the electronic device for calculating physical energy consumption may provide power to the power module such that the power module may provide necessary power to each module of the electronic device for calculating physical energy consumption so as to promote the continuous operation ability of the electronic device for calculating physical energy consumption.

### BRIEF DESCRIPTION OF THE DRAWINGS

FIG. 1 is a first schematic diagram of the first embodiment of electronic device for calculating physical energy consumption of the present invention.

FIG. 2 is a second schematic diagram of the first embodiment of electronic device for calculating physical energy consumption of the present invention.

FIG. 3 is a schematic diagram of the second embodiment of electronic device for calculating physical energy consumption of the present invention.

FIG. 4 is a first schematic diagram of the third embodiment of electronic device for calculating physical energy consumption of the present invention.

FIG. 5 is a second schematic diagram of the third embodiment of electronic device for calculating physical energy consumption of the present invention.

FIG. 6 is a schematic diagram of the fourth embodiment of electronic device for calculating physical energy consumption of the present invention.

### DETAILED DESCRIPTION OF THE PREFERRED EMBODIMENTS

Hereinafter, embodiments of the present invention will be described in detail with reference to the accompanying drawings so that those skilled in the art to which the present invention pertains can realize the present invention. As those skilled in the art would realize, the described embodiments may be modified in various different ways, all without departing from the spirit or scope of the present invention.

The drawings and description are to be regarded as illustrative in nature and not restrictive. Similar reference numerals designate similar elements throughout the specification.

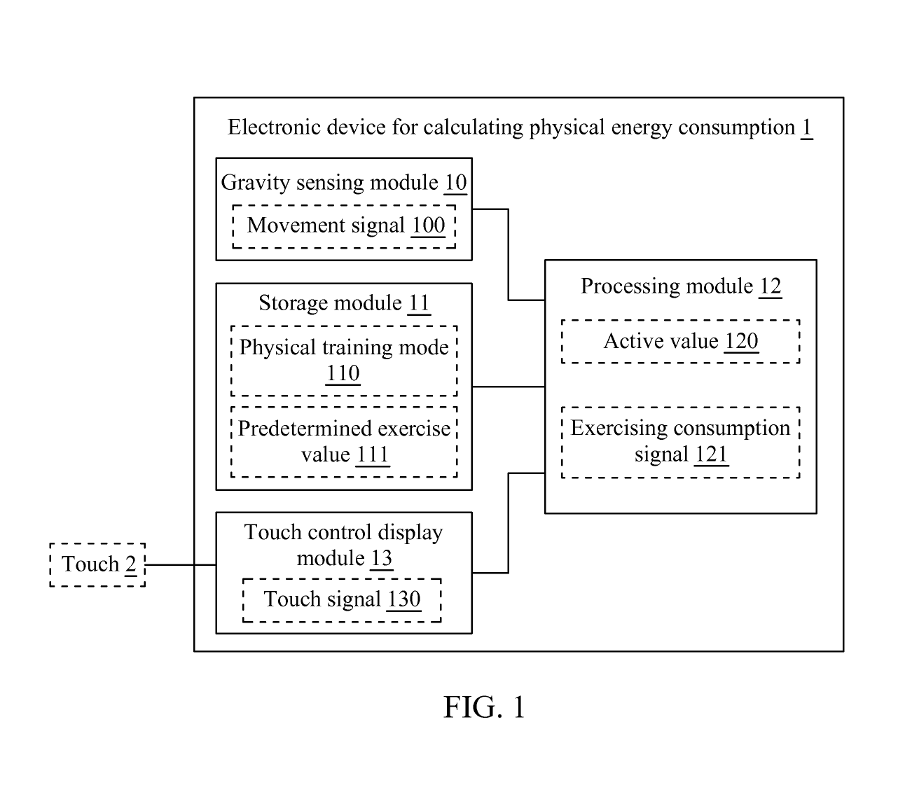

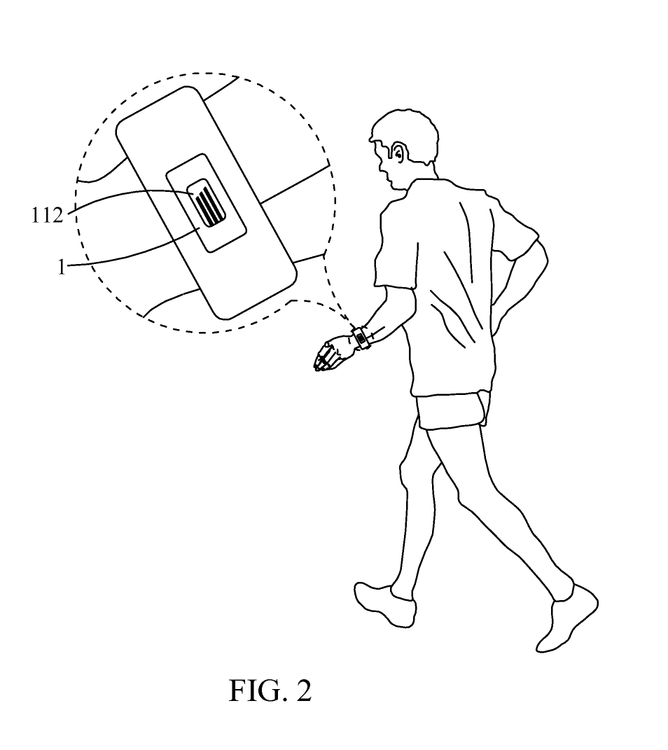

Please refer to FIGS. 1 and 2 which are a first schematic diagram of the first embodiment of electronic device for calculating physical energy consumption of the present invention and a second schematic diagram of the first embodiment of electronic device for calculating physical energy consumption of the present invention, respectively. As the FIGS. show, an electronic device for calculating physical energy consumption 1 comprises a gravity sensing module 10, a storage module 11, a processing module 12, and a touch control display module 13. The gravity sensing module 10 detects at least one movement of the electronic device for calculating physical energy consumption 1 to generate at least one movement signal 100. The storage module 11 stores a plurality of physical training modes 110 and a plurality of predetermined exercise values 111. The processing module 12 is connected to the gravity sensing module 10 and the storage module 11, the processing module 12 receives at least one movement signal 100 and generates at least one active value 120 depending on the movement signal and subtracts at least one active value 120 from the predetermined exercise value 111 corresponding to one of the physical training modes 110 to generate at least one exercising consumption signal 121. The touch control display module 13 is connected to the processing module 12, and the touch control display module 13 receives and displays at least one exercising consumption signal 121.

Specifically, the electronic device for calculating physical energy consumption 1 calculates the user's exercising consumption amount according to the movement generated by the user so as to provide the user for the convenience of acquiring the self-exercise consumption amount and resolve the problems concerning the user has to calculate the self-exercise consumption amount of the prior art. When the user is doing exercise by wearing the electronic device for calculating physical energy consumption 1 of the present invention, the user shakes the electronic device for calculating physical energy consumption 1 so that the electronic device for calculating physical energy consumption 1 generates movement actions, at this time, the gravity sensing module 10 detects the movement of electronic device for calculating physical energy consumption 1 to generate at least one movement signal 100.

Subsequently, the processing module 12 receives the at least one movement signal 100 transmitted by the gravity sensing module 10 and generates at least one active value 120 based on the at least one movement signal 100. And the processing module 12 subtracts the at least one active value 120 from a predetermined exercise value 111 corresponding to one of the physical training modes 110 to generate at least one exercising consumption signal 121. For example, if the user consumes 10 calories of the active value 120 and the predetermined exercise value of 3000 calories whenever doing exercise, the processing module 12 continuously subtracts the 3000 calories by 10 calories, and it shows the user that the amount of exercise of the day has matched up the necessary amount of exercise for a day till the 3000 calories are subtracted completely.

Finally, the processing module 12 transmits the at least one exercising consumption signal 121 to the touch control display module 13 and controls the touch control display module 13 to display the at least one exercising consumption signal 121 for providing the user for the self-exercise consumption amount information.

It is noteworthy to mention that when the user keeps exercising, it keeps the electronic device for calculating physical energy consumption shaking as well as shifting; this movement, the gravity sensing module 10 detects the movement of the electronic device for calculating physical energy consumption 1 and a plurality of movement signals 100 is thereby generated. Relatively, while the processing module 12 is receiving the plurality of movement signals 100, it also generates a plurality of active values 120 so that the predetermined exercise value 111 subtracts the plurality of active values 120. After the predetermined exercise value 111 is completely subtracted by the active value 120, it indicates that the user has finished the necessary self-exercise amount for a day.

Furthermore, the touch control display module 13 of the present invention detects at least one touch 2 to generate at least one touch signal 130, the processing module 12 receives the at least one touch signal 130 to accordingly conduct switching in the plurality of physical training modes 110. Wherein, the plurality of physical training modes 110 may be the training modes such as health, weight reduction, weight loss and so on, and the set exercise amounts for each of the physical training modes 110 are not the same. For example, the exercise consumption amount of the physical training modes 110 for the health mode is smaller than that for the weight reduction mode; and the exercise consumption amount of the physical training modes 110 for the weight reduction mode is smaller than that for the weight loss.

That is to say, the user uses the touch control display module 13 to detect the user's at least one touch 2 and to generate at least one corresponding touch signal 130. Next, the processing module 12 switches one physical training mode 110 to another physical training mode 110 from the plurality of physical training modes 110 according to the at least one touch signal 130.

When the user keeps touching the touch control display module 13, the touch control display module 13 generates the plurality of touch signals 130 according to the user's plurality of touches 2. Responsively, the processing module 12 continuously switches the physical training modes 110 according to the plurality of touch signals 130 in the process of the physical training modes 110.

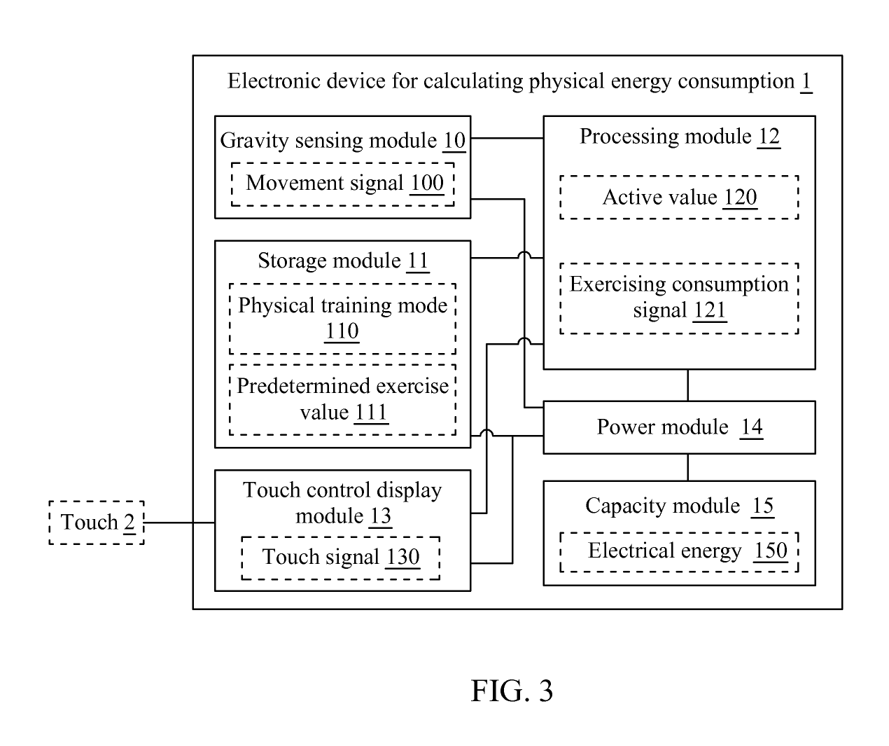

Please refer to FIG. 3 which is a schematic diagram of the second embodiment of electronic device for calculating physical energy consumption of the present invention along with FIG. 1 and FIG. 2. As the FIGS. show, the actions of the electronic device for calculating physical energy consumption of the embodiment are akin to that of the same elements described in the electronic device for calculating physical energy consumption of the first embodiment, and the unnecessary details are therefore not given herein. However, it is worth mentioning that the electronic device for calculating physical energy consumption 1 of the present invention in the embodiment further preferably comprises a power module 14 which is lithium battery connecting to the gravity sensing module 10, the storage module 11, the processing module 12 and the touch control display module 13, and the power module 14 provides the necessary electrical energy for the gravity sensing module 10, the storage module 11, the processing module 12 and the touch control display module 13, and the power module 14.

Moreover, the electronic device for calculating physical energy consumption 1 of the present invention in the embodiment further preferably comprises a capacity module 15 connecting to the power module 14, and the capacity module 15 generates the electrical energy 150 according to the movement of the electronic device, and the capacity outputs the electrical energy 150 to the power module 14.

That is to say, when the power module 14 of the present invention is a rechargeable battery, the electronic device for calculating physical energy consumption 1 is further disposed with the capacity module 15 to recharge to the power module 14. Therefore, when the user is doing exercise by wearing with the electronic device for calculating physical energy consumption 1, the capacity module thereby shacks; simultaneously, the capacity module 15 promptly generates the electrical energy 150 in accordance with the movement of the electronic device for calculating physical energy consumption 1. Afterwards, the capacity module 15 provides the electrical energy 150 to the power module 14 so as to recharge to the power module 14.

Consequently, if the user keeps acting, it enables the capacity module 15 continuously generating the electrical energy 150 such that the capacity module 15 continuously recharges the power module 14; as a result, the electronic device for calculating physical energy consumption 1 is able to extend the running time.

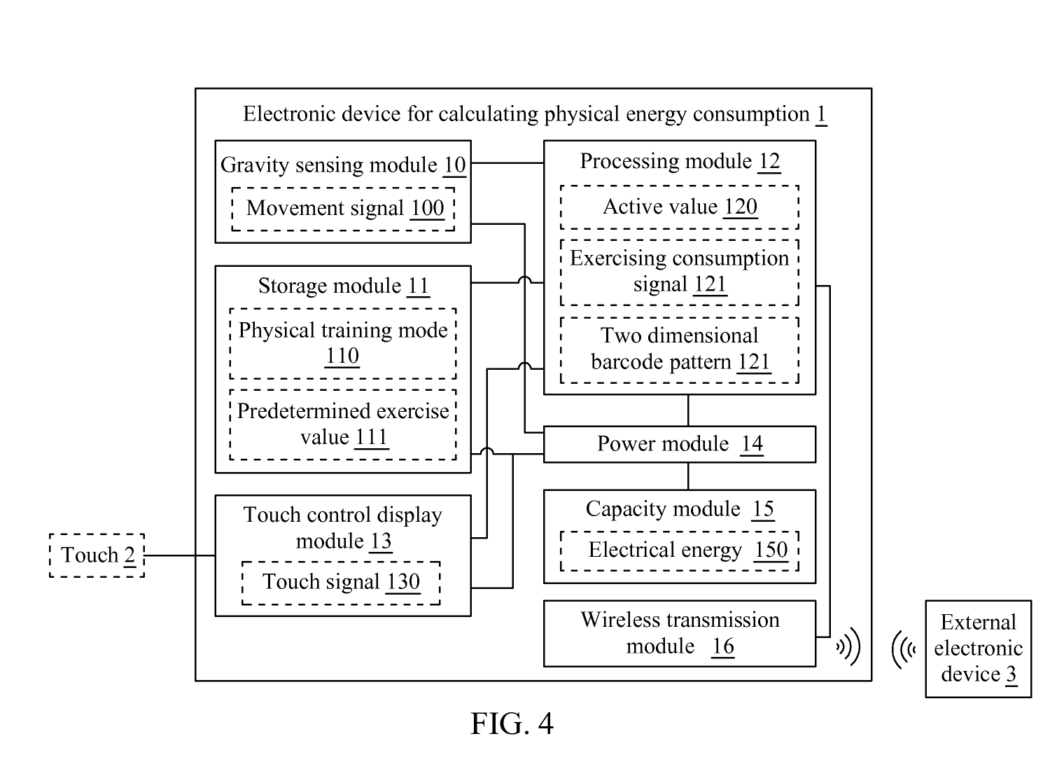

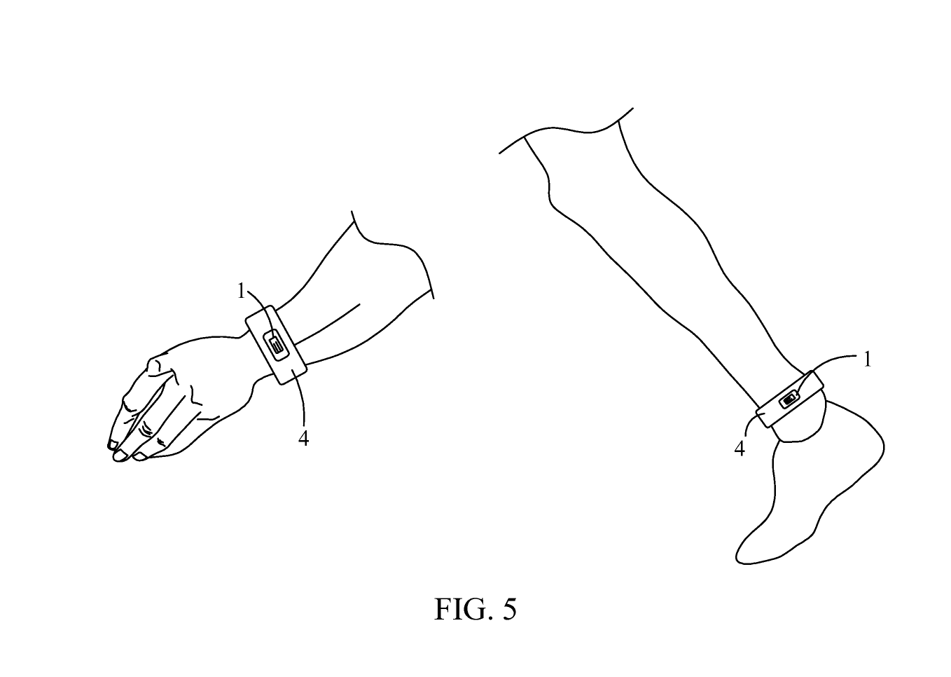

Please refer to FIGS. 4 and 5 which are respectively a first schematic diagram of the third embodiment of electronic device for calculating physical energy consumption of the present invention and a second schematic diagram of the third embodiment of electronic device for calculating physical energy consumption of the present invention along with FIGS. 1 to 3. As the FIGS. show, the actions of the electronic device for calculating physical energy consumption of the embodiment are akin to that of the same elements described in the electronic device for calculating physical energy consumption of the first embodiment, and the unnecessary details are therefore not given herein. However, it is worth mentioning that in the embodiment, the processing module 12 generates a two dimensional barcode pattern 122 according to the exercising consumption signals 121, and the two dimensional barcode pattern 122 is displayed by the touch control display module 13 for image capturing by an external electronic device 3, and the exercising consumption signal 121 is stored in the external electronic device 3.

For example, the processing module 12 of the present invention further conducts coding by numeric value 0-1 of digital signal according to the exercising consumption signals 121 to generate the two dimensional barcode pattern 122 and the two dimensional barcode pattern 122 is displayed via dot matrix display by the touch control display module 13. Subsequently, the user conducts image capturing to the two dimensional barcode pattern 122 displayed on the touch control display module 13 by the external electronic device, such as smart phone, and conducts decoding the two dimensional barcode pattern 122 by software program (i.e. APP) to obtain the exercising consumption signals 121 and to store the exercising consumption signals 121.

By way of the preceding method, the user is able to use the external electronic device 3 to store the exercising consumption signals 121 for benefiting the user from the follow-up statistic data filing or processing.

Furthermore, the electronic device for calculating physical energy consumption 1 of the present invention is detachably connected to an external stationary device 4. Namely, the electronic device for calculating physical energy consumption 1 is detachably clipped or connected to any one external stationary device 4, such as bracelet, foot ring, necklace and so on for benefiting the user wearing on any parts of the body at will, and the effect of portability is thereby accomplished.

Moreover, the electronic device for calculating physical energy consumption 1 further preferably comprises a wireless transmission module 16 connecting to the processing module 12, and the wireless transmission module 16 receives the exercising consumption signal 121 and transmits the exercising consumption signal to the external electronic device 3.

That is to say, the electronic device for calculating physical energy consumption 1 of the present invention is further disposed with the wireless transmission module 16 for providing the user for transmitting the exercising consumption signal 121 to the external electronic device 3 (i.e. personal computer or notebook) via wire transmission mode or the user is able to use wireless transmission mode to transmit the exercising consumption signal 121 to the external electronic device 3 (i.e. smart phone or personal digital assistant). Wherein, the wireless transmission mode of the wireless transmission module 16 may be blue tooth, infrared rays or wireless fidelity.

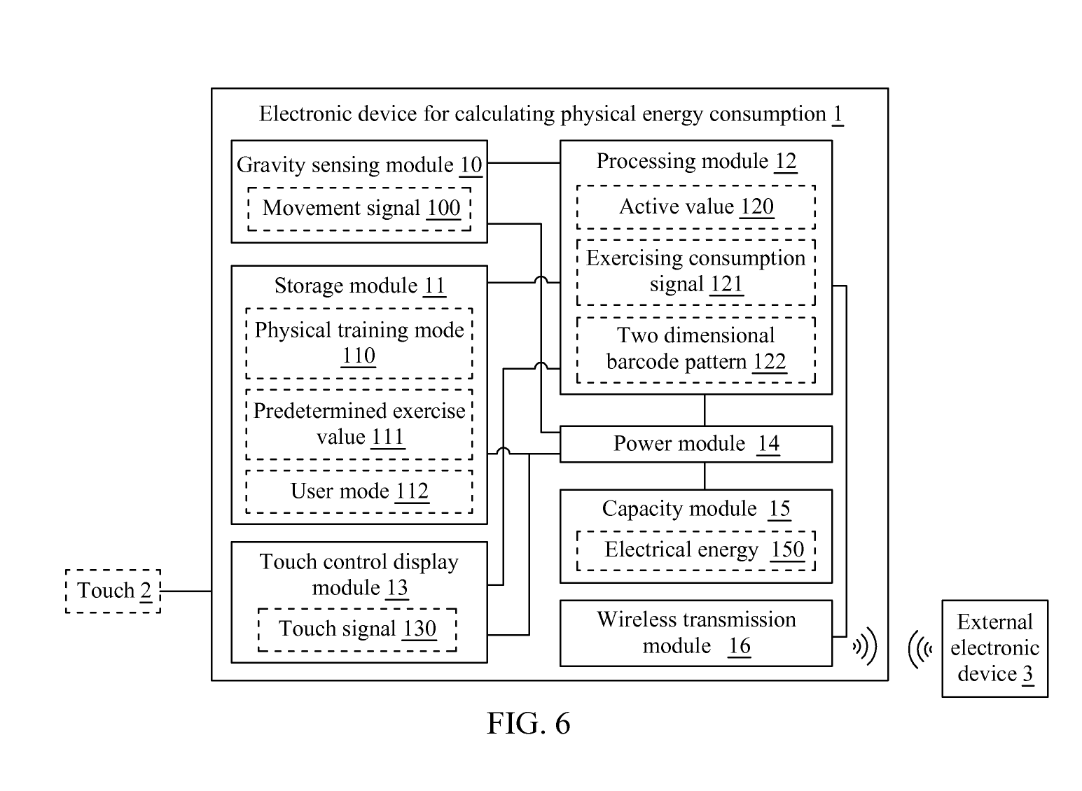

Please refer to FIG. 6 which is a schematic diagram of the fourth embodiment of electronic device for calculating physical energy consumption of the present invention along with FIGS. 1 to 5. As the FIGS. show, the actions of the electronic device for calculating physical energy consumption of the embodiment are akin to that of the same elements described in the electronic device for calculating physical energy consumption of the first embodiment, and the unnecessary details are therefore not given herein. However, it is worth mentioning that in the embodiment, the processing module 11 further stores a plurality of user modes 112 providing setting for the user, and the processing module 12 determines the predetermined exercise value 111 corresponding to each of the physical training modes 110 according to the plurality of user modes 112.

For example, the storage module 11 of the present invention further stores the plurality of user modes 112, and the electronic device for calculating physical energy consumption 1 determines the predetermined exercise value 111 of the physical training modes 110 according to a combination of the setting values of the plurality of user modes 112. That is, the plurality of user modes 112 are the user data such as height, weight, age, gender and so on, respectively. In addition, the processing module 12 determines the predetermined exercise value 111 of the physical training modes 110 according to the combination of the setting values of the plurality of user modes 112.

For instance, in case the user sets the setting values of the user mode 112 by height 175 cm, weight 70 kg, age 24 and gender male, the processing module 12 determines the exercise amount values of the physical training modes 110 of health, weight reduction and weight loss according to the combination of the setting values of the physical training modes 110.

Furthermore, when the control touch display module 13 of the present invention detects the at least one touch 2 and generates the at least one touch signal 130, the processing module 12 receives the least one touch signal 130 to accordingly conduct switching in the plurality of user modes 112. Namely, the user touches the control touch display module 13 to enable the control touch display module 13 detecting the user's at least one touch 2 and to generate the at least one corresponding touch signal 130. Following, the processing module 12 switches one user mode 112 to another user mode 112 from the plurality of user modes 112 according to the at least one touch signal 130.

In addition, it is noteworthy to mention that the electronic device for calculating physical energy consumption 1 of the present invention also further switches to the physical training mode 110 or user mode 112 by the actions of forward tilting or backward tilting of the electronic device for calculating physical energy consumption 1.

And the electronic device for calculating physical energy consumption 1 of the present invention is further disposed with an input module (e.g. button, not shown in FIGS.) to provide the user for switching the modes or each setting value stated in each aforementioned embodiment.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

What is claimed is:

1. An electronic device for calculating physical energy consumption, comprising:
a gravity sensing module detecting at least one movement of the electronic device for calculating physical energy consumption to generate at least one movement signal;
a storage module storing a plurality of physical training modes and a plurality of predetermined exercise values;
a processing module connecting to the gravity sensing module and the storage module, the processing module receiving the at least one movement signal and generating at least one active value depending on the movement signal, and subtracting the at least one active value from the predetermined exercise value corresponding to one of the physical training modes to generate at least one exercising consumption signal; and
a touch control display module connecting to the processing module, and receiving and displaying at least one exercising consumption signal.

2. The electronic device for calculating physical energy consumption of claim 1, wherein the touch control display module detects at least one touch to generate at least one touch signal, and the processing module receives the at least one touch signal to accordingly conduct switching in the plurality of physical training modes.

3. The electronic device for calculating physical energy consumption of claim 1, further comprising a power module connecting to the gravity sensing module, the storage module, the processing module and the touch control display module; the power module providing necessary power to the gravity sensing module, the storage module, the processing module and the touch control display module.

4. The electronic device for calculating physical energy consumption of claim 3, further comprising a capacity module connecting to the power module, the capacity module generating electrical energy according to the movement of the electronic device, and the capacity module outputting the electrical energy to the power module.

5. The electronic device for calculating physical energy consumption of claim 1, wherein the processing module generates a two dimensional barcode pattern according to the exercising consumption signal, and the two dimensional barcode pattern is displayed by the touch control module for image capturing by an external electronic device, and the exercising consumption signal is stored in the external electronic device.

6. The electronic device for calculating physical energy consumption of claim 1, wherein the electronic device for calculating physical energy consumption is detachably connected to an external stationary device.

7. The electronic device for calculating physical energy consumption of claim 1, further comprising a wireless transmission module connecting to the processing module, the wireless transmission module receiving the exercising consumption signal and transmitting the exercising consumption signal to an external electronic device.

8. The electronic device for calculating physical energy consumption of claim 1, wherein the exercising consumption signal is letter, diagram or combination thereof.

9. The electronic device for calculating physical energy consumption of claim 1, wherein the storage module further stores a plurality of user modes providing setting for a user, and the processing module determines the predetermined exercise value corresponding to each of the physical training modes according to the plurality of user modes.

10. The electronic device for calculating physical energy consumption of claim 9, wherein when the touch control display module detects at least one touch and generates at least one touch signal, the processing module receives the at least one touch signal to accordingly conduct switching in the plurality of user modes.
